(19) United States
(12) Patent Application Publication
(10) Pub. No.: US 2010/0331966 A1
(43) Pub. Date: Dec. 30, 2010

(54) BIOCORRODIBLE IMPLANT HAVING AN ACTIVE COATING

(76) Inventor: Alexander Borck, Aurachtal (DE)

Correspondence Address: GREER, BURNS & CRAIN, 300 S WACKER DR, 25TH FLOOR, CHICAGO, IL, 60606, US

(21) Appl. No.: 12/819,484

(22) Filed: Jun. 21, 2010

Related U.S. Application Data
(60) Provisional application No. 61/220,219, filed on Jun. 25, 2009.

(51) Int. Cl. A61F 2/82

(52) U.S. Cl. 623/1.42

(57) ABSTRACT

One embodiment of the invention relates to an implant having a basic body composed of a biocorrodible material and having an active coating or filling of a cavity, consisting of or containing the components
a) at least one pharmaceutically active substance; and
b) an ancillary substance for improving the permeability of the at least one pharmaceutically active substance.

### CROSS REFERENCE This application claims priority on U.S. Provisional Application No. 61/220,219 filed on Jun. 25, 2009.

An aspect of the invention relates to an implant composed of a biocorrodible material, having an active coating or cavity filling that contains at least one pharmaceutical substance.

### BACKGROUND

Medical implants having the most varied purposes are known in great variety from the state of the art. Frequently, only temporary stay of the implant in the body is required to fulfill the medical purpose. Implants composed of permanent materials, in other words materials that are not decomposed in the body, frequently have to be removed again, since over the medium term and also the long term, rejection reactions of the body can occur, even if the biocompatibility is great.

One approach for avoiding another surgical intervention now consists of forming the implant, in whole or in part, from a biocorrodible material. Biocorrosion is understood to be microbial processes, or simply processes caused by the presence of bodily media, that lead to gradual decomposition of the structure that consists of the material. At a certain point in time, the implant or at least the part of the implant that consists of the biocorrodible material loses its mechanical integrity. The decomposition products are absorbed by the body, to a great extent, with slight residues being tolerated.

Biocorrodible materials are developed, among other things, on the basis of polymers of a synthetic nature or of natural origin. However, the material properties, but also, in part, the biocompatibility of the decomposition products of the polymers, clearly limit their use. For example, orthopedic implants frequently have to withstand great mechanical stresses, and vascular implants, e.g. stents, have to satisfy very specific requirements with regard to modulus of elasticity, breaking strength, and shapability, depending on the design.

Stents have the purpose of taking on a supporting function in hollow organs of a patient. For this purpose, stents having a conventional construction have a filigree support structure composed of metallic struts, which is at first present in compressed form, for introduction into the body, and is expanded at the place of application. One of the main areas of use of such stents is the permanent or temporary widening and holding open of vascular constrictions, particularly of constrictions (stenoses) of the coronary blood vessels. In addition, aneurysm stents are also known, which serve to support damaged blood vessel walls. Stents possess a circumferential wall having a sufficient supporting strength so as to hold the constricted blood vessel open to the desired degree, and a tubular basic body through which the blood flow continues without hindrance. The supporting circumferential wall is generally formed by a lattice-like supporting structure that allows introducing the stent, in the compressed state with a small diameter, into the constriction of the blood vessel that is to be treated, in each instance, and expanding it there, for example using a balloon catheter, to such an extent that the blood vessel has the desired, increased inside diameter.

It is furthermore known to make available a pharmaceutically active substance (drug) on the stent, which is particularly supposed to lower the restenosis risk and to support the course of healing. The drug can be applied as a coating, in pure form or embedded into a carrier matrix, or can be made available in cavities of the implant. Examples of suitable drugs include sirolimus and paclitaxel.

One problem in the optimization of stents charged with drugs lies in adjusting the dosage of the drug to be released. In this connection, it is limiting that the amount of the drug to be applied on the outside of the stents is greatly restricted, since the areas on the stent that are available for application are very small. In the case of stents composed of biocorrodible magnesium alloys, the additional problem can occur that the strongly alkaline milieu that results from corrosion of the material can have a negative influence on the absorption behavior of the drug to be taken up. For example, drugs are partly used as hydrochlorides if the solubility of the drug would otherwise be too low. However, such hydrochlorides are transformed back into the deprotonated drug, which has poor solubility, in the strongly alkaline milieu that is formed.

### SUMMARY

One embodiment of the present invention includes an implant having a basic body composed of a biocorrodible material and having one of an active coating and filling of a cavity, comprising: at least one pharmaceutically active substance, and an ancillary substance for improving the permeability of the at least one pharmaceutically active substance.

### DETAILED DESCRIPTION

The present application claims priority on No. 61/220,219 filed on Jun. 25, 2009, which application is incorporated by reference.

Some embodiments of the present invention are based on the task of solving or at least reducing one or more of the problems described above. In particular, absorption of the drugs is supposed to be improved if they are a component of a coating or cavity filling of an implant composed of a biocorrodible material.

This task is accomplished, according to some embodiments of the invention, by means of an implant having a basic body composed of a biocorrodible material, and having an active coating or filling of a cavity consisting of or containing the components
a) of at least one pharmaceutically active substance; and
b) an ancillary substance for improving the permeability of the at least one pharmaceutically active substance.

An ancillary substance for improving the permeability of the at least one pharmaceutically active substance (drug) is understood to be, within the scope of the present invention, compounds that increase penetration of the pharmaceutically active substance through the tissue, and thus increase the tissue concentration of the drug, as the result of chemical, physical, or physiological interactions. Suitable ancillary substances are, for example, benzalkonium chloride, α-tocopherol, glucose, lactose, calcium phosphate, calcium hydrogen phosphate, sodium hydrogen carbonate, sodium carbonate, titanium oxide, zinc oxide, magnesium oxide, silicates such as highly dispersed silicon dioxide (colloidal silicic acid, aerosil, SiO2), talcum, kaolin, bentonite, aliphatic alcohols, DMSO, glycerol, propylene glycol, stearic acid, sugar and sugar alcohols, cyclodextrins such as α-cyclodextrin, β-cyclodextrin, γ-cyclodextrin, as well as mannitol, sorbitol, starches, cellulose powder, cellulose esters and ethers such as methyl cellulose, ethyl cellulose, hydroxyethyl cellulose, hydroxypropyl cellulose, hydroxypropyl methyl cellulose, sodium carboxymethyl cellulose, cellulose acetate phthalate, hydroxypropyl methyl cellulose acetate phthalate, and microcrystalline cellulose, as well as gelatin, gum arabic, pectin, xanthan, alginate, shellac, polyacrylic acids such as carbomer and Eudragit®, as well as polyvinyl pyridine, polyvinyl pyrrolidone, polyvinyl alcohol, polyvinyl acetate, mixtures of polyvinyl pyridine and polyvinyl acetate, polyethylene glycol, vaseline, synthetic and natural fats, silicones, amphiphilic or surfactant ancillary substances such an anion-active tensides, saponides; cationic tensides such as cetyl pyridinium chloride, non-ionogenic tensides; polyoxyethylene sorbitan, macrogol glycerol fatty acid esters, fatty alcohol ethers of polyoxyethylene, fatty acid esters of saccharose, and, in particular, D-α-tocopheryl-1000-succinate, amphoteric tensides, as well as complex emulsifiers such as cetyl stearyl alcohol (Type A and B), quaternary ammonium compounds, preservatives, anti-oxidants such as citric acid, citraconic acid, tartaric acid, monophosphates and polyphosphates, organic phosphates such as dodecyl phosphate, hexose phosphate, as well as butyryl trihexyl citrate (BTHC) and triethyl citrate.

Particularly preferred ancillary substances are hyaluronidases or hyaluronate lyases, derivatives of glycerin, particularly glycerin trilaurate, trimyristate, tripalmitate, and tristearate, as well as polyglycolized glycerides such as Gelucire® 50/13, as well as substances from the group of plasticizers such as tricresyl phosphate (TCP), tributyl phosphate (TBP), acetylated monoglycerides such as alkyl citrate, triethyl citrate, acetyl triethyl citrate, tributyl citrate, acetyl tributyl citrate, trioctyl citrate, acetyl trioctyl citrate, trihexyl citrate, acetyl trihexyl citrate, butyryl trihexyl citrate, trimethyl citrate. These are particularly characterized by clearly improved tissue absorption. In particular, hyaluronidases or hyaluronate lyases are preferred.

Some embodiments of the invention are based on the recognition that the local absorption behavior of active substances, particularly in the strongly alkaline milieu formed during degradation of the stent, can be improved in the presence of hyaluronidases or hyaluronate lyases. The polarity of a large number of neutral active substances is reversed by the alkaline medium. These charged compounds possibly can no longer take on their function, or are no longer able to arrive at their target destination. However, the ancillary substances according to embodiments of the invention have been discovered to allow improved permeability of these possibly impaired active substances. It is furthermore of significant importance that the effect of hyaluronic acid-splitting enzymes is increased in the presence of magnesium ions. This represents a surprising and beneficial result.

Hyaluronic acid is a linear acidic mucopolysaccharide whose structural basic unit is based on a disaccharide that consists of the sugars N-acetyl glucosamine and D-glucuronic acid, which are linked with one another by means of β-1-3 glycoside bonds. These disaccharides in turn are linked with one another, to form a polysaccharide, by way of a β-1-3 glycoside bond. Thus, macromolecules having a molar mass between 200 and 10 000 kDa are formed on the basis of these disaccharide units. On the basis of its structure, hyaluronic acid possesses the capability of forming a large number of hydrogen bridge bonds. Because of this great hydration capacity, hyaluronic acid is one of the structural components in the tissue of vertebrates by way of which the capacity to store water and thus the osmotic equilibrium are controlled.

Because of its degree of swelling, it can also act as a barrier by way of which the flow of ions, proteins, and cells in the tissue is influenced.

Hyaluronic acid-splitting enzymes represent a very comprehensive group of enzymes, on the basis of their variety and complexity, which occur in a great number of organisms in nature, and fulfill some quite different functions there. In mammals, hyaluronidases influence many regulative processes on the cellular level. The hyaluronic acid-splitting enzymes are divided into three large main groups, on the basis of their splitting mechanism, the split location, and the resulting decomposition products.

Hyaluronidases comprise hyaluronate glucanohydrolases and β-1-3 hyaluronate glucanohydrolases. Hyaluronate glucanohydrolases split hyaluronic acid by means of hydrolysis, as endohydrolases, at the β-1-3 glycoside bond, to form saturated oligosaccharides with D-glucuronic acid at the non-reduced and N-acetyl-β-D-glucosamine at the reduced molecule end. β-1-3 hyaluronate glucanohydrolases split hyaluronic acid by means of hydrolysis, as endohydrolases, at the D-glucuronic acid in the β-1-3 position, to form saturated oligosaccharides with N-acetyl-β-D-glucosamine at the non-reduced and D-glucuronic acid at the reduced molecule end.

Another group is represented by hyaluronate lyases, which split hyaluronic acid by means of β-elimination at the β-1-4 glycoside bond at the N-acetyl-β-D-glucosamine, to form oligosaccharides and disaccharides with unsaturated hexuronic acid radicals at the non-reduced molecule end.

Hyaluronidases are used in order to accelerate the speed of tissue absorption after subcutaneous or intramuscular administration of pharmaceutically active substances, or to improve the absorption of medications through intact skin. Since most of the active substances that are administered by subcutaneous or intramuscular administration get into the capillaries by way of the intercellular space, the composition of the basic substance in which the hyaluronic acid dominates plays an important role. If the hyaluronic acid is decomposed by the hyaluronidases, an accelerated spread of the active substance occurs in the interstitium, and thus improved drug absorption is achieved. As a “spreading factor,” it promotes the structural loosening of connective and supportive tissue, and thus promotes the fluid exchange between the tissues and the vascular system. The penetration-promoting properties of the hyaluronidases are particularly applied in local and ophthalmologic anesthesia. Furthermore, hyaluronidase and/or hyaluronate lyase is/are used in topical formulations for accelerating absorption of drugs through intact skin, since topically applied hyaluronidase is able to penetrate the stratum corneum.

In experiments in hyperlipidemic rabbits, concerning the influence of repeated administration of hyaluronidase, it was possible to prove that under the effect of hyaluronidase, lowering of the blood cholesterol and of the fibrin content, an increase in the heparin content in the plasma, an increased vascular permeability in the tissue, and a significant decrease in atheromatous vascular plaque occurred. For this reason, the treatment of vascular diseases in humans, at high dosages of hyaluronidase, is recommended for cardiac arrhythmia, thromboses, cerebral infarctions, cerebral thromboses, and cardiac infarctions. This plaque-loosening effect of hyaluronidase, which is obtained from bovine testes, was also confirmed for bacteria hyaluronate lyase. In addition to direct treatment of infarctions, the damage that occurs as the result of an infarction can be reduced by means of hyaluronidase. For example, hyaluronidase has a positive effect on the collateral blood flow into the ischemic tissue. Hyaluronidase increases the heart-lymph volume and thereby improves the postischemic recovery of heart function. By means of active drainage of the heart lymph with hyaluronidase, the formation of myocardial edemas is reduced, and thus the function of the heart is protected. The proteins released by the myocardium accumulate significantly in the heart lymph, the volume of which gradually increases as a result of the infarction event. This lymph flow can be increased by means of hyaluronidase, thereby preventing occlusion and/or collapse of the lymph vessels. This process could also be supported by the hyaluronidase-stimulated endogenous release of NO, which prevents thrombocyte and leukocyte adhesion and aggregation. Hyaluronidases thus have a positive pharmaceutical effect simply in and of themselves.

The active coating and/or cavity filling contains not only the ancillary substance for improving the permeability of the at least one pharmaceutically active substance, particularly the hyaluronic acid-splitting enzyme, but also at least one pharmaceutically active substance. In particular, this pharmaceutically active substance is selected from the group that comprises antiphlogistics, preferably dexamethasone, methyl prednisolone, and diclophenac; cytostatics, preferably paclitaxel, colchicine, actinomycin D, and methotrexate; immune-suppressives, preferably limus compounds, further preferably sirolimus (rapamycin), myolimus, novolimus, zotarolimus (Abt-578), tacrolimus (FK-506), everolimus, biolimus, particularly biolimus A9 and pimecrolimus, cyclosporin A and mycophenolic acid; thrombocyte aggregation inhibitors, preferably abciximab and iloprost; statins, preferably simvastatin, mevastatin, atorvastatin, lovastatin, pitavastatin, pravastatin, and fluvastatin; estrogens, preferably 17b-estradiol, daidzein and genistein; lipid regulators, preferably fibrates; immune-suppressives; vasodilators, preferably sartans; calcium channel blockers; calcineurin inhibitors, preferably tacrolimus; anti-inflammatory drugs, preferably imidazole; anti-allergics; oligonucleotides, preferably decoy-oligodesoxynucleotide (dODN); endothelium-forming agents, preferably fibrin; steroids; proteins/peptides; proliferation inhibitors; analgesics and antirheumatics; endothelin receptor antagonists, preferably bosentan; rho-kinase inhibitors, preferably fasudil; RGD-peptides and cyclic RGD (cRGD) (comprising the sequence Arg-Gly-Asp); and organic gold or platinum compounds.

The ancillary substance for improving the permeability of the at least one pharmaceutically active substance, particularly the hyaluronic acid-splitting enzyme, and the pharmaceutically active substance can further be embedded in a matrix, preferably composed of a biocorrodible polymer. Biocorrodible or biodegradable polymers particularly comprise polydioxanone; polyorthoesters; polyesteramides; polycaprolactone, polyglycolidene; polylactidene, preferably poly(L-lactide), poly(D-lactide), poly(D,L-lactide), as well as blends, copolymers and tripolymers of them, preferably poly(L-lactide-co-glycolide), poly(D-L-lactide-co-glycolide), poly(L-lactide-co-L-lactide), poly(L-lactide-co-trimethylene carbonate); polysaccharide, preferably chitosan, levan, hyaluronic acid, heparin, dextran, chondroitin sulfate, and celluloses; polyhydroxyvalerate; ethyl vinyl acetate; polyethylene oxides; polyphosphoryl choline; fibrin; albumin; and/or polyhydroxybutyric acids, preferably atactic, isotactic and/or syndiotactic polyhydroxybutyric acid as well as their blends. Also non-degrading or slowly degrading polymers such as: polyphosphazenes such as polyaminophosphazenes or poly[bis(trifluoroethoxy)phosphazene], polyurethanes, such as pellethane, or polyethers and polyether block amides, such as pebax, and polyamides.

Implants in the sense of the invention are devices introduced into the body by way of a surgical procedure, and comprise reinforcement elements for bones, for example screws, plates, or nails, surgical suture material, intestinal clamps, vascular clips, prostheses in the sector of hard and soft tissue, and anchoring elements for electrodes, particularly of pacemakers or defibrillators.

Preferably, the implant is a stent. Stents having a conventional construction demonstrate a filigree structure composed of metallic struts, which is present in a non-expanded state at first, for introduction into the body, and which is then expanded into its expanded state at the place of application. The stent can be coated onto a balloon before or after crimping.

Alloys and elements that undergo decomposition/recomposition in a physiological environment, so that the part of the implant that consists of the material is entirely or at least predominantly no longer present, are referred to as biocorrodible in the sense of the invention. Biocorrodible metallic materials in the sense of the invention comprise metals and alloys selected from the group that comprises iron, tungsten, zinc, molybdenum, and magnesium, and particularly those biocorrodible metallic materials that corrode to form an alkaline product in aqueous solution.

For example, the metallic basic body consists of pure iron, a biocorrodible iron alloy, a biocorrodible tungsten alloy, a biocorrodible zinc alloy, or a biocorrodible molybdenum alloy. Preferably, the metallic basic body consists of magnesium. In particular, the biocorrodible metallic material is a magnesium alloy.

A biocorrodible magnesium alloy is understood to be a metallic structure whose main component is magnesium. The main component is the alloy component whose weight proportion in the alloy is the greatest. A proportion of the main component preferably amounts to more than 50 wt. %, particularly more than 70 wt. %. Preferably, the biocorrodible magnesium alloy contains yttrium and other rare earth metals, since such an alloy is distinguished by its physical chemistry properties and great biocompatibility, particularly also of its decomposition products. A magnesium alloy having the composition rare earth metals 5.2-9.9 wt. %, of that yttrium 3.7-5.5 wt. %, and remainder <1 wt. %, is particularly preferably used, where magnesium makes up the missing part of the alloy, up to 100 wt. %. This magnesium alloy already confirmed its particular suitability in experiments and in first clinical trials, i.e. it demonstrated great biocompatibility, advantageous processing properties, good mechanical characteristics, and a corrosion behavior that was adequate for the purposes of use. In the present case, the general term “rare earth metals” is understood to mean scandium (21), yttrium (39), lanthanum (57) and the 14 elements that follow lanthanum (57), namely cerium (58), praseodymium (59), neodymium (60), promethium (61), samarium (62), europium (63), gadolinium (64), terbium (65), dysprosium (66), holmium (67), erbium (68), thulium (69), ytterbium (70), and lutetium (71).

In its composition, the magnesium alloy in many invention embodiments should be selected in such a manner that it is biocorrodible, although in some embodiments it may not be. Alloys in which decomposition takes place in a physiological environment, which decomposition leads, in the final analysis, to the entire implant or the part of the implant formed from the material losing its mechanical integrity, are referred to as biocorrodible in the sense of the invention. Artificial plasma, as prescribed according to EN ISO 10993-15:2000 for biocorrosion experiments (composition NaCl 6.8 g/l, CaCl₂ 0.2 g/l, KCl0.4 g/l, MgSO₄ 0.1 g/l, NaHCO₃ 2.2 g/l, Na₂HPO₄ 0.126 g/l, NaH₂PO₄ 0.026 g/l) serves as the test medium for testing the corrosion behavior of an alloy being considered. For this purpose, a sample of the alloy to be studied is stored in a sealed sample container, with a defined amount of the test medium, at 37° C. At specific time intervals—coordinated with the expected corrosion behavior—of a few hours to several months, the samples are removed and examined for evidence of corrosion, in known manner. The artificial plasma according to EN ISO 10993-15:2000 corresponds to a blood-like medium and thus represents a possibility for reproducibly presenting a physiological environment in the sense of the invention.

A coating in the sense of the invention is an application, at least in certain sections, of the components to the basic body of the stent. Preferably, the entire surface of the stent is covered by the coating. A layer thickness preferably lies in the range of 1 μm to 100 μm, particularly preferably 3 μm to 15 μm. The coating consists of at least one active substance and the ancillary substance for improving the permeability of the at least one pharmaceutically active substance, particularly the enzyme (hyaluronidase or hyaluronate lyase). The coating can furthermore contain a matrix that accommodates the two components, particularly composed of a biocorrodible polymer. Alternatively, the aforementioned components can be part of a cavity filling. The cavity is situated on the surface or in the interior of the basic body, so that in the latter case, release of the components only takes place after exposure of the cavity as the result of degradation of the basic body. In some embodiments of the invention, then, a cavity forms a concave or other hollowed void space on an implant external surface that may be exposed to the external environment. In other invention embodiments, one or more cavities may be provided in an implant interior. In some cases, the cavity may be completely encapsulated and isolated from the external environment by the implant, so that the cavity interior is only exposed to the external environment after at least some portion of the biocorrodible implant has been corroded to expose the interior. The active substance and ancillary substance can be present in the coating spatially separated from one another, if necessary also in different matrices. Some invention embodiments may include both one or more cavities and a coating.

In a preferred embodiment, the ancillary substance for improving the permeability of the at least one pharmaceutically active substance, particularly hyaluronidase or hyaluronate lyase, is present in the coating in a concentration on the order of an additive, between 0.01 and 2%. Other concentrations will also be useful in other embodiments, and in some cases much higher concentrations are contemplated depending on the application and the particular ancillary substance selected.

An addition on the order of an additive improves the tissue absorption without detrimentally influencing the other mechanical properties of the implant. The addition in the amount described above changes the crystallinity in the case of many drugs, such as paclitaxel, for example, in such a suitable manner that the bioavailability is improved by way of the easier and faster solution capacity at the changed crystallinity.

The invention will be explained in greater detail below, using an exemplary embodiment.

### EXEMPLARY EMBODIMENT 1

### Coating of a Stent

Stents composed of the biocorrodible magnesium alloy WE43 (93 wt. % magnesium, 4 wt. % yttrium (W), and 3 wt. % rare earth metals (E) other than yttrium) are washed with chloroform, rinsed with deionized water, and dried.

The following coating solutions are prepared:

| | |
|---|---|
| Solution A | Solvent chloroform |
| | PLLA (trade name L210 from Boehringer Ingelheim) 1 g/l |
| | Paclitaxel 150 mg/l |
| Solution B | Solvent 100 parts by weight chloroform and parts |
| | by weight methanol |
| | PLLA (trade name L210 from Boehringer Ingelheim) 1 g/l |
| | Hyaluronate lyase (25,000-50,000 IU). |

The solutions are applied to the stent in layers, by spraying them on, and dried. The last layer applied is Solution B.

It will be apparent to those skilled in the art that numerous modifications, equivalents and variations of the described examples and embodiments are possible in light of the above teaching. The disclosed examples and embodiments are presented for purposes of illustration only.

Therefore, it is the intent to cover all such modifications and alternate embodiments as may come within the true scope of this invention.

What is claimed is:

1. An implant having a basic body composed of a biocorrodible material and having one of an active coating and filling of a cavity, comprising the components:
a) at least one pharmaceutically active substance; and
b) an ancillary substance for improving the permeability of the at least one pharmaceutically active substance.

2. The implant according to claim 1, in which the implant is a stent.

3. The implant according to claim 1, in which the biocorrodible material consists of one of magnesium and a magnesium alloy.

4. The implant according to claim 1, in which the ancillary substance is selected from the group consisting of benzalkonium chloride, α-tocopherol, glucose, lactose, calcium phosphate, calcium hydrogen phosphate, sodium hydrogen carbonate, sodium carbonate, titanium oxide, zinc oxide, magnesium oxide, silicates such as highly dispersed silicon dioxide (colloidal silicic acid, aerosil, SiO2), talcum, kaolin, bentonite, aliphatic alcohols, DMSO, glycerol, propylene glycol, stearic acid, sugar and sugar alcohols, cyclodextrins such as α-cyclodextrin, β-cyclodextrin, γ-cyclodextrin, as well as mannitol, sorbitol, starches, cellulose powder, cellulose esters and ethers such as methyl cellulose, ethyl cellulose, hydroxyethyl cellulose, hydroxypropyl cellulose, hydroxypropyl methyl cellulose, sodium carboxymethyl cellulose, cellulose acetate phthalate, hydroxypropyl methyl cellulose acetate phthalate, and microcrystalline cellulose, as well as gelatin, gum arabic, pectin, xanthan, alginates, shellac, polyacrylic acids such as carbomer, as well as polyvinyl pyridine, polyvinyl pyrrolidone, polyvinyl alcohol, polyvinyl acetate, mixtures of polyvinyl pyridine and polyvinyl acetate, polyethylene glycol, vaseline, synthetic and natural fats, silicones, amphiphilic or surfactant ancillary substances such as anion-active tensides, saponides; cationic tensides such as cetyl pyridinium chloride, non-ionogenic tensides; polyoxyethylene sorbitan, macrogol glycerol fatty acid ester, fatty alcohol ether of polyoxyethylene, fatty acid ester of saccharose, and, in particular, D-α-tocopheryl-1000-succinate, amphoteric tensides, as well as complex emulsifiers such as cetyl stearyl alcohol (Type A and B), quaternary ammonium compounds, preservatives, anti-oxidants such as citric acid, citraconic acid, tartaric acid, monophosphates and polyphosphates, organic phosphates such as dodecyl phosphate, hexose phosphate, hyaluronidases or hyaluronate lyases, derivatives of glycerin as well as glycerin trilaurate, trimyristate, tripalmitate, and tristearate, as well as polyglycolized glycerides, as well as substances from the group of plasticizers such as tricresyl phosphate (TCP), tributyl phosphate (TBP), acetylated monoglycerides such as alkyl citrate, triethyl citrate, acetyl triethyl citrate, tributyl citrate, acetyl tributyl citrate, trioctyl citrate, acetyl trioctyl citrate, trihexyl citrate, acetyl trihexyl citrate, butyryl trihexyl citrate, and trimethyl citrate.

5. The implant according to claim 4, in which the ancillary substance is one of hyaluronidases and hyaluronate lyases.

6. The implant according to claim 1, in which the components a) and b) are embedded in a matrix composed of a biocorrodible polymer.

7. The implant according to claim 1, in which the pharmaceutically active substance is selected from the group consisting of antiphlogistics, cytostatics, immune-suppressives, thrombocyte aggregation inhibitors, estrogens, lipid regulators, immune-suppressives; vasodilators, sartans; calcium channel blockers; calcineurin inhibitors, anti-inflammatory drugs, anti-allergics; oligonucleotides, endothelium-forming agents; steroids; proteins/peptides; proliferation inhibitors; analgesics and antirheumatics; endothelin receptor antagonists, rho-kinase inhibitors, RGD-peptides and cyclic RGD (cRGD) (comprising the sequence Arg-Gly-Asp); and organic gold or platinum compounds.

8. An implant according to claim 7 wherein:
the antiphlogistics are one or more of dexamethasone, methyl prednisolone, and diclophenac;
the cytostatics are one or more of paclitaxel, colchicine, actinomycin D, and methotrexate;
the immune-suppressives are one or more of limus compounds, sirolimus (rapamycin), myolimus, novolimus, zotarolimus (Abt-578), tacrolimus (FK-506), everolimus, biolimus, biolimus A9 and pimecrolimus, cyclosporin A, and mycophenolic acid;
the thrombocyte aggregation inhibitors are one or more of abciximab and iloprost; statins, simvastatin, mevastatin, atorvastatin, lovastatin, pitavastatin, pravastatin, and fluvastatin;
the estrogens are one or more of 17b-estradiol, daidzein and genistein;
the lipid regulators are fibrates;
the vasodilators are sartans;
the calcineurin inhibitors are tacrolimus;
the anti-inflammatory drugs are imidazole;
the oligonucleotides are decoy-oligodesoxynucleotide (dODN);
the endothelium-forming agents are fibrin;
the endothelin receptor antagonists are bosentan; and,
the rho-kinase inhibitors are fasudil.

9. An implant according to claim 1, where the component b) is present in one of the coating and filling of a cavity in a concentration between 0.01 and 2%.

10. An implant according to claim 1 wherein the components a) and b) fill the one or more cavities.

11. An implant according to claim 10 wherein at least one of the one or more cavities are provided in a basic body interior wherein release of the components a) and b) only takes place after the basic body degrades and the interior cavity is exposed.

12. An implant according to claim 1 wherein the components a) and b) are provided in a coating that covers at least a portion of the implant basic body.

13. An implant according to claim 1 wherein the components a) and b) are provided in different matrices and are spatially separated from one another.

14. An implant according to claim 13 wherein the coating completely covers at least an exterior surface of the basic body and is provided in a thickness between about 3 to about 15 μm.

15. An implant according to claim 1 wherein the basic body is made of a magnesium alloy having a composition of at least about 70 wt. % magnesium and between about 5.2-9.9 wt % rare earth metals that include at least yttrium.

16. A stent comprising:
a biocorrodible basic body consisting of one of iron, an iron alloy, a tungsten alloy, a zinc alloy, a molybdenum alloy, and a magnesium alloy;
a material carried by the basic body, having at least one pharmaceutically active substance and an ancillary substance for improving the permeability of the at least one pharmaceutically active substance, the ancillary substance comprising one or more of hyaluronidase and hyaluronate lyase present in the material in a concentration of between about 0.01 and about 2 wt %.

17. The stent according to claim 16 wherein the material is provided in a coating that overlies at least a portion of the basic body surface, the coating having a thickness of between about 1 to about 100 μm.

18. The stent according to claim 16 wherein the basic body defines at least one cavity, and wherein the material is provided within the cavity.

19. The stent according to claim 16 wherein the basic body defines at least one internal cavity, and wherein the material is provided within the internal cavity wherein it will be exposed only after the biocorrodible body has decomposed.

20. The stent according to claim 16 wherein:
the material is carried in a biocorrodible matrix; and,
the basic body consists essentially of a biocorrodible magnesium alloy in which magnesium comprises at least 50 wt % and in which one or more rare earth metals comprise up to about 9.9 wt %.
